(19) United States
(12) Patent Application Publication
(10) Pub. No.: US 2023/0168518 A1
(43) Pub. Date: Jun. 1, 2023

(54) SYSTEM AND METHOD FOR MONITORING AND REGULATING THE LEVEL OF THE CONTENTS IN A BIOCONTAINER

(71) Applicant: Merck Patent GmbH, Darmstadt (DE)

(72) Inventors: Laurent Jourdainne, Stutzheim-Offenhein (FR); Stefano Berti Perez, Boston, MA (US)

(21) Appl. No.: 17/997,296

(22) PCT Filed: Jun. 1, 2021
(86) PCT No.: PCT/EP2021/064607

§ 371 Date: Oct. 27, 2022

(30) Foreign Application Priority Data
Jun. 4, 2020 (EP) 20305590.0

(51) Int. Cl. G02B 27/10; C12M 1/00; C12M 1/06; C12M 1/21; C12M 1/34; G01F 23/292

(52) U.S. Cl. CPC G02B 27/108; C12M 23/14; C12M 23/28; C12M 27/02; C12M 41/02; C12M 41/44; G01F 23/2921

(57) ABSTRACT

A system and a method for monitoring and regulating the level of contents in a vessel is disclosed. In some embodiments, the system or the method use laser beams and photosensors to determine the level of the contents of the vessel.

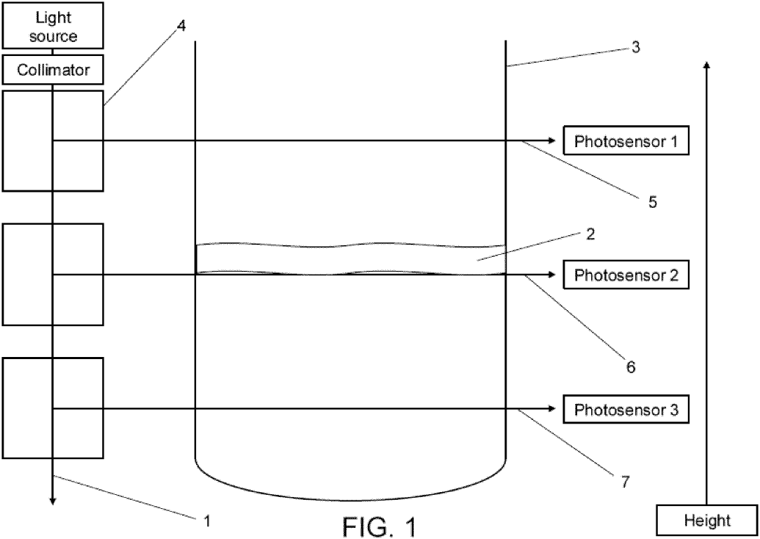

### RELEVANT FIELD

Embodiments disclosed herein relate to bioprocessing. More specifically, some embodiments of the technology relate to monitoring and regulating the level of contents within a biocontainer. In some embodiments, level detection is performed by lasers and photosensors.

### BACKGROUND

Foam occurs in bioprocessing partially due to the introduction of gases into the culture medium. Foaming during bioprocessing leads to reduced productivity resulting from bursting bubbles damaging valuable products, a loss of sterility if the foam escapes the bioreactor, or overpressure if the foam blocks an exit filter. Chemical antifoaming agents, also referred to as “antifoams”, “de-foaming agents”, or “defoamers,” are routinely used in bioreactors to reduce the amount of foam during bioprocessing. Antifoam agents are known also to negatively affect the processes taking place in the bioreactor. Therefore, a system is needed to monitor the amount of foam in the bioreactor and to add antifoam only when the monitoring shows the amount of foam exceeds a safe level.

Present mechanical and visual level detection systems do not provide a clear in situ solution to achieve foam detection. Visual detection systems frequently detect foam with simple camera technology. Using vision level detection systems can be expensive and requires developing an algorithm for image processing for foam or level recognition. Software development is a long and complex process with low added value for only foam or level detection. Also, the software required for live analysis of the image cannot be embedded in many user services platform (USP) software or demand side platform (DSP) software frequently used in the art.

Mechanical level detection systems are inexpensive and widely used, but the systems are intrusive. Further, the localization is stationery within the bioreactor. In some cases, radar or ultrasonic probes are placed inside the bioreactor, but introducing a foreign object increases the risk of contamination of the contents of the bioreactor, fouling of the probes by foam residues, and leaks.

A non-intrusive system with an optical sensor based on laser light detection to monitor the level of liquid or foam in a biocontainer and sound an alarm when critical levels are reached, which is based on a versatile implementation, represents an inventive advance in the art.

### SUMMARY

The shortcomings of the prior art are overcome by embodiments described herein, which include some embodiments disclosed herein providing an optical sensor system for a vessel, the system comprising: a light source capable of emitting laser light through the vessel; a beamsplitter capable of splitting the laser light into more than one beam, wherein each of the more than one beam is at a different height; and more than one photosensor capable of measuring the light intensity of each beam, wherein each more than one photosensor corresponds to one laser beam to form an optical channel.

In some embodiments, the vessel is a bioreactor. In some embodiments, the vessel further comprising a mixer. In some embodiments, the mixer is capable of being used in upstream bioprocessing applications. In some embodiments, the system is single-use. In some embodiments, the system is capable of measuring the level of contents within the vessel. In some embodiments, the contents within the vessel comprise a liquid. In some embodiments, the liquid comprises a solution. In some embodiments, the contents within the vessel comprise foam. In some embodiments, the contents within the vessel comprise air. In some embodiments, the photosensor is capable of differentiating among the light intensity detected after passing through air, foam, or liquid within the vessel. In some embodiments, the system described herein further comprises a collimator. In some embodiments, the system further comprises an alarm capable of being activated by the contents reaching a critical level within the vessel. In some embodiments, the photosensor is a photodiode.

In some embodiments, the system is external to the vessel. In some embodiments, the vessel is transparent or translucent. In some embodiments, the vessel is stainless steel and further comprises at least two windows.

Some embodiments described herein provide a method of preventing overfilling or overpressure within a vessel during bioprocessing, the method comprising: splitting a laser light into at least two beams, wherein the at least two beams comprise a first beam and a second beam; directing the first beam through a level of the vessel representing the maximum fill level for contents of the vessel; wherein the maximum fill level is higher than the level of the contents prior to beginning or continuing bioprocessing; directing the second beam through a level of the vessel representing a level for the contents of the vessel; monitoring continuously the light intensity of the at least two beams by detecting with at least two photosensors, wherein the at least two photosensors comprise a first photosensor and a second photosensor with the first photosensor measuring the light intensity of the first beam and the second photosensor measuring the light intensity of the second beam; activating of an alarm when a decrease in light intensity is detected by the first photosensor compared to the light intensity detected by the first photosensor prior to an increase in the level of contents in the vessel; and reducing the level of contents in the vessel in response to the alarm, whereby preventing overfilling or overpressure within the vessel.

In some embodiments, the vessel is a biocontainer. In some embodiments, the biocontainer is a single-use bioreactor bag. In some embodiments, the contents of the vessel comprise liquid. In some embodiments, the contents of the vessel comprise foam. In some embodiments, the method further comprises reducing the level of the contents in the vessel by adding an anti-foaming agent to the contents of the vessel.

### BRIEF DESCRIPTION OF THE DRAWINGS

The FIGURE provides an illustration of some embodiments of the level monitoring and regulation system for a biocontainer described herein.

The appended drawings illustrate some embodiments of the disclosure herein and are therefore not to be considered limiting in scope, for the invention may admit to other equally effective embodiments. It is to be understood that elements and features of any embodiment may be found in other embodiments without further recitation and that, where possible, identical reference numerals have been used to indicate comparable elements that are common to the figures.

### DETAILED DESCRIPTION

The disclosure herein describes some embodiments of a system for level monitoring and regulation in a vessel, such as a biocontainer. For example, a biocontainer with a mixer within upstream bioprocessing applications. In some embodiments, the biocontainer is a bag or a bioreactor.

A laser-based sensor system has the potential to be less expensive, modular, and scalable compared to a vision-based system for monitoring and regulating the level of contents in a biocontainer. In some embodiments, the system described herein is modular, i.e., the laser-based system can be successfully implemented with a control device communicating therewith based on a variety of software platforms. In some embodiments, the term modular describes the characteristic of the system described herein as being compatible with different types of bioreactors known in the art. For example, the system may be compatible with single-use bioreactors. Alternatively, the system may be compatible with stainless steel bioreactors comprising at least two windows capable of allowing a laser to pass through the internal cavity of the bioreactor.

Also, in some embodiments, the data processing behind the laser-based sensor system described herein can be simply and fully implementable in presently used software platforms, such as USP software and DSP software.

Some embodiments of the system comprise a non-intrusive sensor, which need not be placed within the inner volume of the biocontainer and does not contact the contents of the biocontainer. Some embodiments are part of a single-use detection system. The application can either be foam or liquid level monitoring and regulation to prevent an excess accumulation of foam, overfilling, or overpressure inside the vessel.

### I. System Components

The Figure is an illustration of some embodiments of the monitoring and regulation system described herein. In some embodiments, a light source emits a laser light 1 with a defined wavelength through a vessel 3. In some embodiments, the vessel 3 is a biocontainer or a bioreactor. In some embodiments, the bioreactor holds volumes of up to 10 L or more, specifically with a total volume of approximately 0.35, 1.5, 5.0, 10 L with a working volume ranging between about 700 and 1300 ml, about 1 to 3 L, or about 2.5 to 10 L. In some embodiments, the bioreactor holds a volume of up to about 100 L, about 200 L, about 500 L, about 1000 L, about 2000 L, about 2500 L, or about 3000 L. In some embodiments, the bioreactor is bench scale (e.g. about 3 L). For example, the vessel 3 is a Mobius® 3L Single-use Bioreactor (MilliporeSigma).

In some embodiments, the bioreactor is a multiple-use or reusable bioreactor. In some embodiments, the multiple-use bioreactor comprises stainless steel. In some embodiments, the bioreactor is a single-use bioreactor. In some embodiments, the bioreactor comprises or consists of a material conforming to the United States Pharmacopeia (USP) Class VI requirements, such as a plastic material. The plastic material may be selected from polyamide, polycarbonate, polymethylpentene, or polystyrene. The disposable bioreactor may be formed of monolayer or multilayer flexible walls of a polymeric composition such as polyethylene, for example, ultra-high molecular weight polyethylene, linear low density polyethylene, low density or medium density polyethylene, polypropylene, ethylene vinyl acetate (EVOH), polyvinyl chloride (PVC), polyvinyl acetate (PVA), ethylene vinyl acetate copolymers (EVA copolymers), blends of various thermoplastics, co-extrusions of different thermoplastics, multilayered laminates of different thermoplastics, or the like as described in the patent families of US 10,675,836 and WO 2019/199406, each of which is hereby incorporated by reference in its entirety. “Different” is meant to include different polymer types such as polyethylene layers with one or more layers of EVOH as well as the same polymer type but of different characteristics such as molecular weight, linear or branched polymer, fillers, and the like.

Typically, medical grade and preferably animal-free plastics are used, which are generally are sterilizable such as by steam, ethylene oxide, or radiation, such as beta or gamma radiation. Most have good tensile strength, low gas transfer, and are either transparent or at least translucent. In some embodiments, the material is weldable or gluable to form a fluid tight connection with other features of a bioreactor and is unsupported.

In some embodiments, welding techniques can be selected from the group consisting of plastic welding or heat sealing, for example, ultrasonic welding, laser welding, welding using infra-red radiation, or thermal welding. In some embodiments, the material is clear or translucent, allowing visual monitoring of the contents. In some embodiments, the bioreactor is integrally formed in an injection molding process or a blow molding process.

In some embodiments, the bioreactor is a disposable, deformable, and/or foldable bag defining an inner volume, that is sterilizable for a single use, capable of accommodating contents, such as biopharmaceutical fluids, in a fluid state, and that can accommodate a mixing device partially or completely within the inner volume. In some embodiments, the inner volume can be opened, such as by suitable valving, to introduce a fluid into the volume, and to expel fluid therefrom, such as after mixing is complete. In some embodiments, the bioreactor may be a two-dimensional or “pillow” bag, or the bioreactor may be a three-dimensional bag. The particular geometry of the bioreactor is not limited. In some embodiments, the bioreactor includes a rigid base, which provides access points to the inner volume, such as ports or vents.

In some embodiments, the laser light 1 is split into several beams located at different heights (e.g., the three beams 5, 6, and 7) using a beamsplitter 4. In some embodiments, a beam 5 is located at a height equivalent to the maximum level for the contents of the vessel 3 to prevent overfill or overpressure of the vessel 3. In some embodiments, a beam 6 is located at a height at or near the foam or liquid level 2 of the contents of the vessel 3 prior to beginning or continuing bioprocessing. In some embodiments, a beam 7 is located at a height resulting in the beam 7 travelling through liquid contents of the vessel 3.

In some embodiments, the laser light 1 is split into at least two beams. In some embodiments, the laser light 1 is split into at least three beams. For example, the laser light 1 is split into three, four, five, six, seven, eight, or nine beams. In some embodiments, the laser light 1 is split as often as desired. In some embodiments, several light sources are used to generate the more than one beam.

In some embodiments, the wavelength of the laser light 1 is within the range of 780 nm to 900 nanometers (nm). For example, the wavelength of the laser light is selected from about 780 nm, about 790 nm, about 800 nm, about 810 nm, about 820 nm, about 830 nm, about 840 nm, about 850 nm, about 860 nm, about 870 nm, about 880 nm, about 890 nm, or about 900 nm. In some embodiments, the wavelength of the laser light 1 is 780 nm to be close to turbidity standard wavelength (800 nm). In some embodiments, the beam (any of beams 5, 6, and 7) has an elliptical shape section of about 1 mm². In some embodiments, the elliptical shape section of the beam (5, 6, and 7) is less than 1 mm². For example, the elliptical shape section of the beam (5, 6, and 7) is about 0.1, about 0.2, about 0.3, about 0.4, about 0.5, about 0.6, about 0.7, about 0.8, or about 0.9 mm².

In some embodiments, the system comprises more than one beamsplitter 4. For example, the system comprises two, three, four, five, six, or seven beamsplitters 4. In some embodiments, the system comprises three beamsplitters 4. In some embodiments, the beamsplitter 4 is a non-polarizing cube.

In some embodiments, the beamsplitter 4 has a beam diameter within the range of about 3 millimeters (mm) to about 150 mm. In some embodiments, the beamsplitter has a beam diameter of about 5 mm. For example, the beam diameter is selected from the group consisting of 3 mm, 4 mm, 5 mm, 6 mm, 7 mm, and 8 mm. In some embodiments, the beamsplitter has a reflectance/transmittance (R/T) ratio adjustable between 10/90, 30/70, 50/50, 70/30, and 90/10, and all ranges therebetween. In some embodiments, the beamsplitter 4 accepts wavelengths within the range of between 700 nm to 1100 nm. In some embodiments, the wavelength is at least one range selected from the group consisting of: the range of 675 nm to 750 nm, the range of 725 nm to 800 nm, the range of 775 nm to 850 nm, the range of 825 nm to 900 nm, the range of 875 nm to 950 nm, the range of 925 nm to 1000 nm, the range of 975 nm to 1050 nm, the range of 1025 nm to 1100 nm, and the range of 1075 nm to 1150 nm. In some embodiments, the wavelength is about 700 nm, about 725 nm, about 750 nm, about 775 nm, about 800 nm, about 825 nm, about 850 nm, about 875 nm, about 900 nm, about 925 nm, about 950 nm, about 975 nm, about 1000 nm, about 1025 nm, about 1050 nm, about 1075 nm, about 1100 nm, or about 1125 nm. In some embodiments, each beam is paired with a photosensor to form an optical channel. In some embodiments, two or more optical channels measure continuously or at the same time. In some embodiments, the optical channels are identical to the each other except for the height localization of each. For example, the laser wavelength is the same for each optical channel. In some embodiments, each optical channel includes the same type of photosensor. In some embodiments, the height localization of each channel is free before any sensor manufacturing and can be driven by the details of the application of the system.

In some embodiments, an optical channel is formed by one beam (e.g., 5, 6, or 7) and one photosensor located on a same theoretical diameter of the vessel 3, resulting in the incident light being fully perpendicular to the circular shape of the vessel 3 (normal incidence) to avoid any refraction. Therefore, the transmitted light is measured by a photosensor, such as a photodiode.

In some embodiments, the photosensor is a silicon-based photodiode. Alternatively, in some embodiments, the photosensor is a photodiode comprising at least one material selected from the group consisting of germanium, indium gallium arsenide, lead(II) sulfide, and mercury cadmium telluride. In some embodiments, the photodiode was capable of detecting wavelengths between 320 nm to 1100 nm. For example, the wavelength detected by the photodiode is within at least one range selected from the group consisting of: 300 nm to 400 nm, 350 nm to 450 nm, 400 nm to 500 nm, 450 nm to 550 nm, 500 nm to 600 nm, 550 nm to 650 nm, 600 nm to 700 nm, 650 nm to 750 nm, 700 nm to 800 nm, 750 nm to 850 nm, 800 nm to 900 nm, 850 nm to 950 nm, 900 nm to 1000 nm, 950 nm to 1050 nm, 1000 nm to 1100 nm, 1050 nm to 1150 nm, and 1100 nm to 1200 nm. In some embodiments, the photodiode is cathode grounded

### II. Operating the Regulation System

Advantages of some embodiments of the system described herein are the ability to use a bench-top optical component, easy to process data and signal, and less cost than traditional optical and mechanical systems in the prior art.

In some embodiments, one mode of operation is integration of a two optical channel laser-based sensor system as described herein into USP equipment. In some embodiments, one optical channel including a beam 6 is located at the liquid surface level (foam channel) 2. In some embodiments, a second optical channel including a beam 5 is located at a reasonable distance from the top of the bag (top channel). In some embodiments, this dual optical channel arrangement of the system functions as a critical level sensor. In some embodiments, an increase in the level of foam 2 in the vessel 3 is indicated by a decrease in the light intensity of the beam 5 detected by a photosensor. Once the light intensity of the beam 5 is under a threshold value, the foam has reached a specific height in the vessel. Then, the level information is fed into the regulation loop for monitoring the biocontainer.

In some embodiments, the regulation loop is managed by a control device, such as a microprocessor or computer, connected to a power supply, which provides power to the light source(s). In some embodiments, when the control device receives information the level of contents in the biocontainer has reached a critical level, the control device triggers release of an anti-foaming agent from a conduit in fluid communication with an internal cavity of a single-use or stainless steel biocontainer or bioreactor.

In some embodiments, the following four measurements situations occur in a system with at least two optical channels:

1) Light intensities of greater than 0 mA are measured in both optical channels, which means the level of the contents in the biocontainer is low or no foam is detected.

2) A light intensity of 0 mA is measured in the foam channel, and a light intensity of greater than 0 mA is measured in the top channel. These results mean foam or opaque solution is present, but no overflow of the contents has occurred. In some embodiments, anti-foaming agent is added for regulation of the level of the contents in the biocontainer.

3) A light intensity of greater than 0 mA is measured in the foam channel, and a light intensity of 0 mA is measured in the top channel. For a transparent solution, the foam level is too high, and a critical alarm may be activated.

4) When 0 mA light intensity is measure in both optical channels, the foam or liquid level is too high in the biocontainer. In some embodiments, a critical alarm is activated. In some embodiments, an anti-foam agent is added to the contents of the biocontainer.

### III. Definitions

Unless defined otherwise, all technical and scientific terms used herein have the same meaning as commonly understood by one of ordinary skill in the art to which this invention pertains.

The term, “biocontainer” or “bioreactor,” as used herein, refers to any manufactured or engineered device or system that supports a biologically active environment and are used interchangeably herein. In some instances, a bioreactor is a vessel in which a cell culture process is carried out which involves organisms or biochemically active substances derived from such organisms. Such a process may be either aerobic or anaerobic. Commonly used bioreactors are typically cylindrical, ranging in size from liters to cubic meters, and are often made of stainless steel. In some embodiments described herein, a bioreactor is made of a material other than steel and is disposable or single-use. In some embodiments, the biocontainer is a bag. It is contemplated that the total volume of a bioreactor may be any volume ranging from 100 mL to up to 10,000 liters or more, depending on a particular process.

The term “bioprocessing,” as used herein, refers to any application of the biological systems of living cells or their components, such as bacteria, enzymes, or chloroplasts, to obtain a target product. In some embodiments, bioprocessing takes place in a biocontainer, such as a bioreactor. Bioprocessing may encompass upstream and downstream bioprocessing. Upstream bioprocessing includes cell culture.

The term, “critical level,” as used herein, refers to a level of the contents within the bioreactor beyond which the bioreactor will be over-filled or over-pressured resulting in failure of the bioreactor. In some embodiments, the height of a foam within the bioreactor or biocontainer represents the critical level.

The terms, “laser” or “light source,” as used herein, refers to a device capable of producing a coherent beam of light.

The term, “mixer,” as used herein, refers to a component of a bioreactor including an agitator capable combining the components used within bioprocessing methods and processes.

The term, “photosensor,” as used herein, refers to a device capable of both detecting light and measuring the light intensity of a beam. The term photosensor may include, for example, an electronic component that detects the presence of visible light, infrared transmission (IR), and/or ultraviolet (UV) energy.

As used herein, the singular forms “a”, “an,” and “the” include plural unless the context clearly dictates otherwise.

### EXAMPLES

### Example 1. Differentiating Materials

The sensitivity of a photodiode used as a photosensor were analyzed by measuring light intensities as a beam passed through different conditions within a bioreactor. An elliptical beam laser diode at 780 nm (2.5 mW) was used as a light source. The laser diode was contained in 0.8 mm housing with a collimator integrated into the housing. Transmitted light intensities from the light source were measured by a silicon (Si)-based photodiode as the photosensor. The mounted Si-based photodiode was capable of detecting wavelengths between 320 nm to 1100 nm and was cathode grounded. Results for different conditions in a Mobius® 3L Single-use Bioreactor (MilliporeSigma) are shown in Table 1.

TABLE 1

Light Intensities Measured under Various Conditions

| | |
|---|---|
| Condition | Intensity [mA] |
| In the air (baseline) | 302 mA |
| Through the vessel + air (above the solution) | 273 mA |
| Through vessel + solution | 184 mA |
| Through vessel + thick foam | 0 mA |
| Through vessel + light foam | 29 mA |

The results in Table 1 provide show the photodiode was slightly sensitive to the amount of foam, and the photodiode was sensitive enough to the optical index of the medium to differentiate among the different conditions. Therefore, the system was observed to discriminate among air, foam, and solution in the vessel.

### EQUIVALENTS

All ranges for formulations recited herein include ranges therebetween and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, ... 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4, or 3.1 or more.

Reference throughout this specification to “one embodiment,” “certain embodiments,” “one or more embodiments,” “some embodiments,” or “an embodiment” indicates that a feature, structure, material, or characteristic described is included some embodiments of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment.

Publications of patent applications and patents and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. An optical sensor system for a vessel, the system comprising:
a light source capable of emitting laser light through the vessel;
a beamsplitter capable of splitting the laser light into more than one beam, wherein each of the more than one beam is at a different height of the vessel; and
more than one photosensor capable of measuring the light intensity of each beam, wherein each more than one photosensor corresponds to one beam to form an optical channel.

2. The system of claim 1, wherein the vessel is a bioreactor or biocontainer.

3. The system of claim 1, the vessel further comprising a mixer.

4. The system of claim 3, wherein the mixer is capable of being used in at least one of upstream bioprocessing applications and downstream bioprocessing applications.

5. The system of claim 1, wherein the system is single-use.

6. The system of claim 1, wherein the system is capable of measuring the level of contents within the vessel.

7. The system of claim 6, wherein the contents within the vessel comprise a liquid.

8. The system of claim 7, wherein the liquid comprises a solution.

9. The system of claim 6, wherein the contents within the vessel comprise foam.

10. The system of claim 6, wherein the contents within the vessel comprise air.

11. The system of claim 8, wherein the photosensor is capable of differentiating among the light intensity detected after passing through air, foam, or liquid within the vessel.

12. The system of claim 1, further comprising a collimator.

13. The system of claim 6, further comprising an alarm capable of being activated by the contents reaching a critical level within the vessel.

14. The system of claim 1, wherein the photosensor is a photodiode.

15. The system of claim 1, wherein the system is external to the vessel.

16. The system of claim 1, wherein the vessel is transparent or translucent.

17. The system of claim 1, wherein the vessel is stainless steel and further comprises at least two windows.

18. A method of preventing overfilling or overpressure within a vessel during bioprocessing, the method comprising:
splitting a laser light into at least two beams, wherein the at least two beams comprise a first beam and a second beam;
directing the first beam through a level of the vessel representing the maximum fill level for contents of the vessel; wherein the maximum fill level is higher than the level of the contents prior to beginning or continuing bioprocessing;
directing the second beam through a level of the vessel representing a level for the contents of the vessel;
monitoring continuously the light intensity of the at least two beams by detecting with at least two photosensors, wherein the at least two photosensors comprise a first photosensor and a second photosensor with the first photosensor measuring the light intensity of the first beam and the second photosensor measuring the light intensity of the second beam;
activating of an alarm when a decrease in light intensity is detected by the first photosensor compared to the light intensity detected by the first photosensor prior to an increase in the level of contents in the vessel; and
reducing the level of contents in the vessel in response to the alarm, whereby preventing overfilling or overpressure within the vessel.

19. The method of claim 18, wherein the vessel is a biocontainer.

20. The method of claim 19, wherein the biocontainer is a single-use bioreactor bag.

21. The method of claim 18, wherein the contents comprise liquid.

22. The method of claim 18, wherein the contents comprise foam.

23. The method of claim 18, wherein reducing comprises adding an anti-foaming agent to the contents of the vessel.
